(19) United States
(12) Patent Application Publication
(10) Pub. No.: US 2006/0252030 A1
(43) Pub. Date: Nov. 9, 2006

(54) Fabrication of carbohydrate chips by immobilizing unmodified carbohydrates on derivatized solid surfaces, and their uses

(76) Inventors: Shin In-Jae, Kyungki-do (KR); Lee Myung-Ryul, Kyungki-do (KR)

Correspondence Address: CANTOR COLBURN, LLP, 55 GRIFFIN ROAD SOUTH, BLOOMFIELD, CT, 06002, US

(21) Appl. No.: 11/190,340

(22) Filed: Jul. 27, 2005

(30) Foreign Application Priority Data
May 6, 2005 (KR) 10-2005-0038077

(51) Int. Cl. C12Q 1/70; G01N 1/28; G01N 33/554; G01N 33/00

(52) U.S. Cl. 435/5; 435/7.32; 436/90; 427/2.11; 438/1

(57) ABSTRACT

The present invention relates to the fabrication of carbohydrate chips wherein free carbohydrates are immobilized on a modified solid surface. More specifically, it relates to a fabrication method for carbohydrate chips wherein unmodified carbohydrates irrespective of their size are site-specifically and covalently attached to the solid surface derivatized by hydrazide or aminooxy groups. According to the present method, unmodified carbohydrates are efficiently immobilized on the solid surface regardless of their size, and carbohydrate chips are easily fabricated because the method does not require modified carbohydrates. Furthermore, the invention relates to the use of the fabricated carbohydrate chips for the rapid analysis of carbohydrate-protein interactions and the diagnosis of carbohydrate-based diseases.

### FIELD OF THE INVENTION

The present invention relates to the fabrication of carbohydrate chips wherein free carbohydrates are immobilized on a modified solid surface. More specifically, it relates to a fabrication method for carbohydrate chips wherein unmodified carbohydrates irrespective of their size are site-specifically and covalently attached to the solid surface derivatized by hydrazide or aminooxy groups. Furthermore, the invention relates to their uses for the rapid analysis of carbohydrate-protein interactions and the diagnosis of carbohydrate-based diseases by detecting pathogens, cancers and so on.

### BACKGROUND OF THE INVENTION

Functional studies of carbohydrates, called as functional glycomics, have received considerable attention for biological research and biomedical applications. The cell surface is highly covered with diverse structures of glycans, mainly present in the forms of glycoconjugates such as glycoproteins and glycolipids. The cell surface carbohydrates are involved in a variety of important biological processes, including cell communication, cell adhesion, fertilization, development, differentiation, and immune response through specific interactions with proteins. These interactions are also involved in detrimental disease processes. For example, it is through these interactions that bacteria or viruses adhere to host cells and confer pathogenic properties. In addition, tumor metastasis and inflammation happen through carbohydrate-protein recognition events, too. Therefore, carbohydrates or carbohydrate-binding proteins specifically found in tumor cells or pathogens are often utilized as markers for their diagnoses. As a consequence, elucidation of the molecular basis for glycan-protein interactions aids development of potent biomedical agents such as anti-cancer agents, antibiotics, anti-viral agents and anti-inflammatory agents, as well as development of new diagnosis to detect cancers and pathogens.

Details of carbohydrate-protein interactions have been conventionally investigated by biophysical or biochemical approaches.

Although these techniques have largely contributed to understanding of these interactions, these are not suitable for the rapid analysis of the interactions between proteins and carbohydrates. Thus, there has been a need for carbohydrate chips which are capable of simultaneously examining a number of samples within a short period of time [Shin, I. et al., Chem. Eur. J. 2005, 11, 2894; Shin I. et al., Combinatorial Chemistry and High-Throughput Screening 2004, 7, 565; Feizi, T. et al., Curr. Opin.

Struct. Biol. 2003, 13, 637].

The carbohydrate chips have been applied to biological research and biomedical applications: 1) high-throughput analysis of glycan-protein interactions, 2) rapid characterization of carbohydrate-processing enzymes such as glycosidases, glycosyltransferases, and so on, 3) quantitative determination of binding affinities between carbohydrates and proteins, 4) high-throughput screening of inhibitors which suppress carbohydrate-protein interactions, 5) analysis of glycans attached to glycoproteins, and 6) diagnosis of cancers or pathogens.

Conventional methods for the fabrication of carbohydrate chips are 1) to site-specifically and covalently immobilize modified carbohydrates onto properly derivatized solid surface [see FIG. 1(a)], 2) to site-specifically but noncovalently immobilize neoglycolipids on the underivatized solid surface [see FIG. 1(b)], and 3) to nonspecifically and noncovalently immobilize unmodified carbohydrates on the underivatized solid surface [see FIG. 1(c)]. The first two approaches exhibited the efficient immobilization of carbohydrates on the surface, but need modified carbohydrates whose synthesis is sometimes very difficult and time-consuming. The third method is suitable for the construction of polysaccharide chips, but has the disadvantage of inefficient attachment of small carbohydrates such as mono-, di- or oligosaccharides.

Thus, a more efficient method to overcome these limitations for the preparation of carbohydrate chips is required for their various applications. The present inventors have developed a method to efficiently immobilize a variety of unmodified carbohydrates irrespective of their size on the solid surface derivatized by hydrazide or aminooxy groups [see FIG. 1(d)], to fabricate carbohydrate chips that can be utilized for rapidly assessing carbohydrate-protein interactions and detecting pathogens and cancers, accordingly.

### SUMMARY OF THE INVENTION

The object of the present invention is to provide a fabrication method of carbohydrate chips wherein a variety of unmodified carbohydrates can be site-specifically and covalently immobilized regardless of their size, and to use them for biological research and biomedical applications by rapidly analyzing carbohydrate-protein interactions.

The fabrication of the carbohydrate chips comprises the steps of (i) the modification of the solid surface to which carbohydrates are attached, (ii) printing unmodified carbohydrates on the derivatized solid surface, and (iii) reacting said carbohydrates with the modified solid surface to site-specifically and covalently immobilize the carbohydrates onto the solid surface. The fabricated carbohydrate chips are used to rapidly analyze carbohydrate-protein interactions and to diagnose carbohydrate-based diseases by detecting pathogens, cancers and so on.

The present invention also provides a detection method of proteins bound to carbohydrates immobilized on the surface prepared by the above method, which comprises labeled or non-labeled detection systems.

Further, the present invention provides a method for diagnosing carbohydrate-based diseases using the carbohydrate chips fabricated according to the present invention.

### BRIEF DESCRIPTION OF THE DRAWINGS

FIG. 1 is a schematic diagram showing fabrication methods of carbohydrate chips;

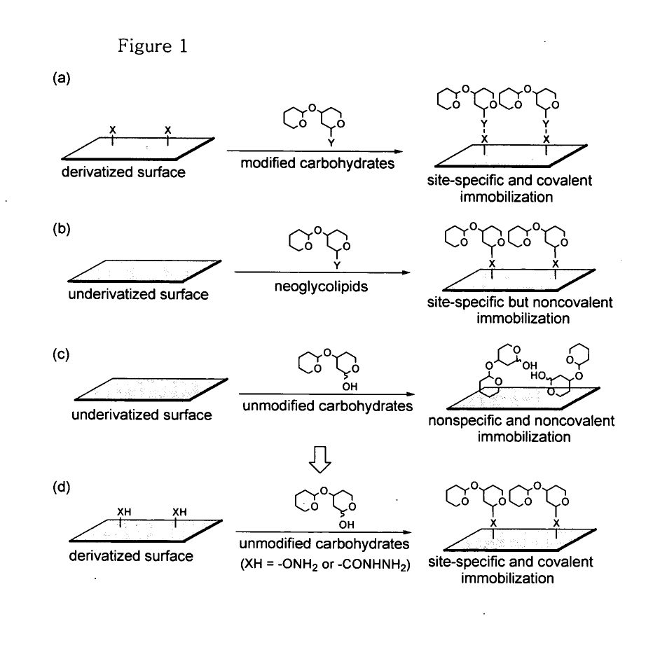

FIG. 2 is a schematic diagram illustrating the principle of the present invention;

FIG. 3 is a flow chart showing the preparation of solid surface derivatized by (a) aminooxy or (b) hydrazides via linkers of various lengths;

FIG. 4 is a graph showing time-dependence of immobilization of fucose and N,N′-diacetylchitobiose on the solid surface coated by aminooxy (dark line) and hydrazides (red line), after incubation with (a) Cy5-labeled AA and (b) Cy3-labeled TV; and

FIG. 5 is a fluorescent image of carbohydrate chips composed of 21 carbohydrates probed with (a) Cy3-TV, (b) Cy5-AA, (c) FITC-ConA, (d) anti-Sialyl Le^{x} antibody and (e) E. Coli pre-incubated with propidium iodide (PI).

### DESCRIPTION OF THE PREFERRED EMBODIMENTS

According to the present invention, functional groups of step (i) are preferably hydrazide and aminooxy groups.

According to the present invention, the solid surface is preferably selected from the group consisting of silicon, polymers, mica, plastics, glass, gold, paper, membrane, and a combination thereof.

The unmodified carbohydrates are natural or chemically or enzymatically prepared carbohydrates, which can be preferably selected from mono-, di-, oligo- and polysaccharides, wherein the monosaccharides are more preferably selected from the group consisting of glucose (Glc), N-acetylglucosamine (GlcNAc), glucuronic acid (GlcA), galactose (Gal), N-acetylgalactosamine (GalNAc), mannose (Man), N-acetylmannosamine (ManNAc), fucose (Fuc), rhamnose (Rham) and xylose (Xyl); the disaccharides are more preferably selected from the group consisting of maltose, cellobiose, N,N′-diacetylchitobiose, lactose, galactose-β1,4-N-acetylglucosamine (Galβ1,4GlcNAc; LacNAc) and mannose-α1,6-mannose (Manα1,6Man; mannobiose); the oligosaccharides are more preferably selected from the group consisting of Fucα1,3(Galβ1,4)Glc (FucLac), NeuNAcα2,3Galβ1,4GlcNAc (NeuNAcLacNAc), sialyl Le^{x} and Fucα1,2Galβ1,3(Fucα1,4)GlcNAcβ1,3Gal β1,4Glc; the polysaccharide is more preferably mannan.

According to the present invention, printing of said unmodified carbohydrates in step (ii) is preferably carried out by using a micropipette or microarrayer.

According to the present invention, the process for site-specific and covalent attachment of said carbohydrates to the solid surface in step (iii) is preferably performed by incubating the printed plates between 40° C. and 60° C. for 8 hours or more.

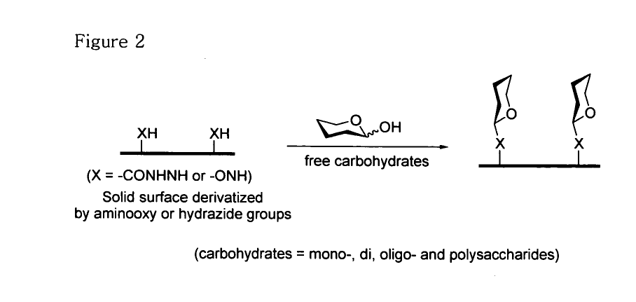

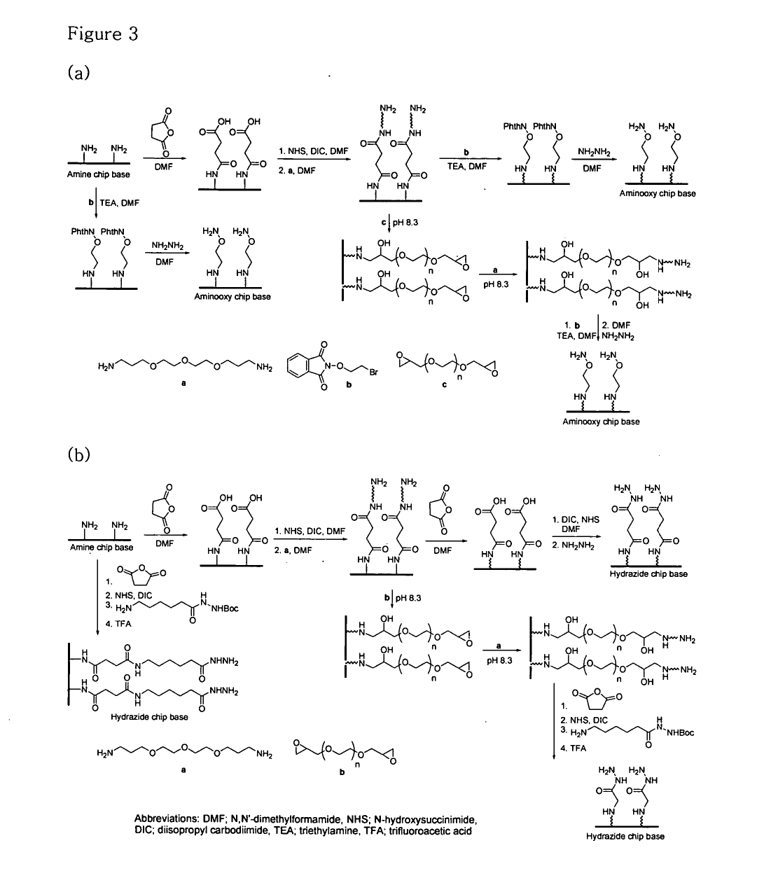

FIG. 2 illustrates the principles of the invention. First, a chip base coated by hydrazide or aminooxy groups via linkers of various lengths is prepared as shown in FIG. 3.

The linkers connected to the hydrazide or aminooxy groups on the solid surface are preferably selected from the group consisting of hydrocarbon chains, preferably non-branched chains, inserted by heteroatoms, and of a length exceeding 10 atoms, preferably containing from 25 to 55 atoms.

More specifically, an aminooxy-derivatized chip base is prepared by reacting an amine chip base with a compound b or a longer linker a or c followed by coupling with a compound b, and then by treating with hydrazine to introduce hydrazide groups on the surface [see FIG. 3(a)], while a hydrazide chip base is prepared either by reacting the amine chip base with 6-aminohexanoic acid hydrazide or a longer linker a or b, followed by removal of protecting groups [see FIG. 3(b)].

A solution (0.1 μl˜1 nl) of carbohydrates including mono-, di-, oligo- and polysaccharides in PBS buffer (pH 4.0-5.0) containing 30-50% glycerol is printed on the above-mentioned chip base by using a micropipette or microarrayer. In this process, the concentration of carbohydrates is preferably between 0.1 mM and 50 mM. After completion of printing, the chip is reacted at 40-60° C. for 8 hours or more, and then washed. Then, the chip is immersed in PBS buffer (pH 7.4) containing 0.1% Tween 20 and 1% BSA (bovine serum albumin) for 1 hour. The fabrication of carbohydrate chips is complete by washing the BSA-treated chip with buffer (pH 7.4) containing 0.1% Tween 20 three times for 15 minutes.

The fabricated carbohydrate chips are applied for studies on protein-carbohydrate interactions. For these studies, the carbohydrate chips are incubated with non-labeled or fluorophore-labeled proteins in buffer containing 0.1% Tween 20 for 1 hour. Proteins labeled by fluorescent dyes such as Cy3, Cy5 and FITC are commercially available, or can be prepared by reacting proteins with fluorescent dyes. Then, the protein-treated chips are washed with buffer containing 0.1% Tween 20 three times for 10 minutes in order to remove unbound proteins.

According to one embodiment of the aspect of the present invention, the binding patterns between proteins and carbohydrates immobilized on the surface are analyzed by labeled or non-labeled detection systems. Non-labeled detection systems are selected from the group consisting of mass spectrometer and surface plasmon resonance imager. Labeled detection systems are selected from the group consisting of scanning-based instruments and instruments using a CCD camera. The sample is human or animal tissue or body fluid including blood serum, urine, milk, sweat, bone marrow, and lymphatic fluid.

According to another aspect of the present invention, provided is a method to diagnose carbohydrate-based diseases by employing the carbohydrate chips fabricated by the above-mentioned pathways. The diseases which can be diagnosed include genetic disorders, cancers, and viral and bacterial infections.

### EXAMPLES

The present invention is now described in more detail by the following examples and the described embodiments are to be considered in all respects only as illustrative and not restrictive.

### Example 1

### Selection of Functional Groups Required for Immobilization of Unmodified Carbohydrates on the Solid Surface

The present inventors initially selected functional groups which were reacted with unmodified carbohydrates with high efficiency and selectivity. The reactions between unmodified carbohydrates and aminooxy or hydrazide groups have been widely used for the synthesis of various glycoconjugates [Hatanaka, Y. et al., J. Org. Chem. 2000, 65, 5639; Leteux, C. et al., Glycobiology 1998, 8, 227; Zhao, Y. et al., Proc. Natl. Acad. Sci. US.A. 1997, 94, 1629]. Accordingly, the present inventors used these reactions for site-specific and covalent attachment of unmodified carbohydrates to the solid surface.

### Example 2

### Preparation of Aminooxy and Hydrazide-Modified Chip Bases

The solid surface modified by aminooxy and hydrazide groups is prepared as shown in FIG. 3. The procedure is described in detail below.

The aminooxy chip bases are prepared according to three processes as follows [see FIG. 3(a)]. First, the aminooxy chip base connected by a short linker is prepared by reacting an amine chip base with a compound b and triethylamine (TEA) for 12 hours, and then with 3% hydrazine for 3-6 hours to remove phthaloyl (Phth) protecting groups. Second, the preparation of the aminooxy chip base connected by a linker a (4,7,10-trioxa-1,3-tridecanediamine) is initiated by reacting the amine chip base with succinic anhydride for 3 hours. The carboxylic acid chip base thus prepared is reacted with N-hydroxysuccinimide (NHS) and diusopropyl carbodiimide (DIC) for 3 hours, and then with a linker a for 3 hours to introduce amine groups onto the surface. The long-tethered amine chip base thus prepared is reacted with a compound b for 12 hours, and then with hydrazine for 3-6 hours to provide an aminooxy chip base connected by the linker of a moderate length. Third, the preparation of the longest aminoxy chip base connected by linkers a and c (polyethylene glycol diglycidyl ether) is initiated by reacting the amine chip base appended by a linker a with a compound c at pH 8.3 for 1 hour to incorporate epoxide therein. The resulting epoxide chip base is reacted with a linker a at pH 8.3 for 3 hours. The amine chip base thus prepared is reacted with a compound b followed by treatment with hydrazine to provide the aminooxy chip base connected by the linker of the longest length.

The hydrazide chip bases are prepared according to three processes as follows [see FIG. 3(b)]. First, an amine chip base connected by a short linker is prepared by sequential reactions with succinic anhydride for 3 hours, NHS and DIC for 3 hours, t-butyloxycarbonyl 6-aminohexanoic acid hydrazide for 3 hours, and trifluoroacetic acid (TFA) for 1 hour. Second, the hydrazide chip base connected by a linker of a moderate length is prepared by using the amine chip base tethered by a linker a described in the preparation of aminooxy chip bases. The amine chip base is reacted with succinic acid to introduce carboxylic acids onto the surface, which are further reacted with DIC and NHS followed by reaction with hydrazine for 3 hours to afford the hydrazide chip base conneted by a linker of a moderate length. Third, the longest hydrazide chip base is prepared from epoxide-coated chip base described above. The epoxide chip base is reacted with a linker a, and then sequentially with succinic anhydride, NHS and DIC, t-butyloxycarbonyl 6-aminohexanoic acid hydrazide, and TFA to produce the longest hydrazide chip base.

The reason why the linkers of various lengths are incorporated into chip bases is to find out a way to minimize steric hindrance and nonspecific interactions during protein binding to the carbohydrate ligands on the solid surface.

### Example 3

### Optimization of Immobilization Conditions

The experiments are carried out to optimize conditions (temperature, time, pH and concentration) for immobilizing carbohydrates on the solid surface derivatized by aminooxy and hydrazide groups prepared in Example 2.

### <3-1>Optimal Immobilization Temperature and Time

In order to optimize immobilization temperature and time, a solution of fucose and N,N′-diacetylchitobiose (30 mM, pH 5.0 sodium phosphate buffer containing 30% glycerol) is printed on the solid surface modified by aminooxy or hydrazide groups, and the resulting chip is incubated at 22° C., 37° C. or 50° C. After 1 to 21 hours, the chip is incubated with the labeled Aleuria aurantia (Cy5-AA) and Triticum vulgaris (Cy3-TV, also known as wheat germ agglutinin) or non-labeled proteins.

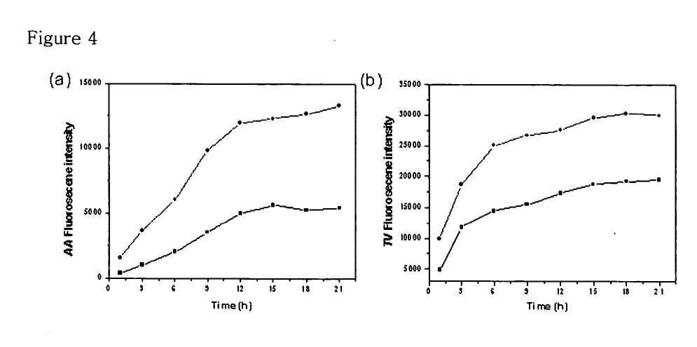

The binding intensities between proteins and carbohydrates on the surface were determined by using a fluorescence scanner or a fluorescence microscopy in the case of labeled proteins and a surface plasmon resonance imager or a mass spectrometer in the case of non-labeled proteins. The carbohydrate chips prepared at 50° C. show the best result among the tested temperatures. In addition, the carbohydrate chips obtained from greater than 12 hour incubation at 50° C. exhibit no substantial change of fluorescence intensities (see FIG. 4). Thus, the immobilization time of about 12 hours is optimal for immobilization.

### <3-2>Optimal Immobilization pH and Concentration

The optimal pH and concentration for immobilization are examined at the optimal temperature (50° C.) and time (12 hours) obtained from Example <3-1>.

Specifically, it is found that conditions of a carbohydrate concentration of about 30 mM concentration and pH 4-5 are ideal for efficient immobilization. It is also found that the covalent linkage between carbohydrates and aminooxy or hydrazide groups on the solid surface is very stable, based on the observation that the extensive washing of the carbohydrate chips with buffer does not affect lectin binding. Further, the aminooxy and hydrazide chip bases connected by the longest linker prepared according to the third process in Example 2 show the best results in comparison with the chip bases tethered by the shorter linkers.

### Example 4

### Applications of Carbohydrate Chips

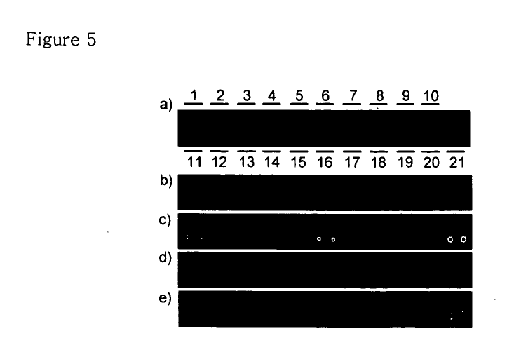

Carbohydrate chips are used for the rapid assessment of carbohydrate-protein interactions, which are involve in various biological processes and are of importance for the development of novel therapeutics, and for the fast diagnosis of carbohydrate-based diseases such as tumors and pathogens. For this purpose, each solution (sodium phosphate buffer containing 30% glycerol, pH 5.0) of twenty one of mono-, di-, oligo- and polysaccharides listed in Table 1 was printed on the solid support coated by aminooxy or hydrazide groups. The carbohydrate chips prepared from the method described in Example 3 were incubated with Cy3-TV, Cy5-AA and FITC-ConA. According to the fluorescence intensity of the spots in chips after detecting with a fluorescence scanner, TV strongly is bound to N,N′-diacetylchitobiose (13), less strongly to GlcNAc (2) and sialyl Le^{x} (19), and weakly bound to GalNAc (5), LacNAc (15) and NeuNAcLacNAc (18) [see FIG. 5(a)]. The carbohydrate chips treated with AA show that the lectin is strongly bound to Fuc (8), FucLac (17) and hexasaccharides (20), but very weakly bound to sialyl Le^{x} (19) [see FIG. 5(b)]. The carbohydrate chips incubated with ConA exhibit the strong binding of the lectin to mannan (21), less strong binding to mannobiose (16), and very weak binding to maltose (11) [see FIG. 5(c)].

Sialyl Le^{x} is an important biological recognition marker and an interesting target for drug discovery. The glycans bearing sialyl Le^{x} on glycoproteins in blood bind to selectins on T cells, endothelial cells or platelets. This binding event causes acute and chronic inflammation (Somers, W. S. et al., Cell 2000, 103, 467; Wild M. K. et al., J. Biol. Chem. 2001, 276, 31602). In addition, sialyl Le^{x} is known to be one of tumor-associated carbohydrate antigens (Hakamori, S. Adv. Cancer Res. 1989, 52, 257). Thus, the detection of sialyl Le^{x} and development of inhibitors for sialyl Le^{x}-binding proteins are important for both basic biological research and drug discovery. Carbohydrate chips prepared by an above-mentioned method were used to detect sialyl Le^{x}-binding proteins. The carbohydrate chips were incubated with anti-sialyl Le^{x} antibody. The fluorescence image of the chip shows that sialyl Le^{x} is selectively recognized by this antibody [see FIG. 5(d)].

Many bacteria including pathogens express specific carbohydrate-binding proteins on pili. The initial attachment of pathogens to host cells through specific carbohydrate-protein interactions confers pathogenic properties. For example, a mannose-binding protein of type 1 fimbriated E. coli is known to cause common urinary tract infection through its binding to glycans on host cells (Connell, H. et al., Proc. Natl. Acad. Sci. USA 1996, 93, 9827). Thus, carbohydrate chips were also used to detect pathogens for diagnosis. E. coli ORN178, expressing mannose-binding adhesin on pili, binds to spots containing mannose, mannobiose and mannan [see FIG. 5(e)].

TABLE 1
Carbohydrates used for the fabrication of carbohydrate chips

| | |
|---|---|
| Monosaccharide | 1. Glc |
| | 2. GlcNAc |
| | 3. GlcA |
| | 4. Gal |
| | 5. GalNAc |
| | 6. Man |
| | 7. ManNAc |
| | 8. Fuc |
| | 9. Rham |
| | 10. Xyl |
| Disaccharide | 11. Maltose |
| | 12. Cellobiose |
| | 13. N,N′-diacetylchitobiose |
| | 14. Lactose |
| | 15. Galβ1,4GlcNac (LacNAc) |
| | 16. Manα1,6Man (Mannobiose) |
| Oligosaccharide | 17. Fucα1,3(Galβ1,4)Glc (FucLac) |
| | 18. NeuNAcα2,3Galβ1,4GlcNAc |
| | (NeuNAcLacNAc) |
| | 19. Sialyl Le^{x} |
| | 20. Fucα1,2Galβ1,3(Fucα1,4)GlcNAc |
| | β1,3Galβ1,4Glc |
| Polysaccharide | 21. Mannan |

As described above, the present inventors provide a novel and efficient fabrication method for carbohydrate chips by site-specifically and covalently immobilizing unmodified carbohydrate on a derivatized solid support. Protein and cell-binding experiments using carbohydrate chips fabricated by this method demonstrate that any type of carbohydrates, regardless of their size, can be efficiently immobilized on the solid surface derivatized by aminooxy or hydrazide groups. In addition, it is showed that carbohydrate chips are useful for the rapid analysis of carbohydrate-protein interactions and the detection of antibodies and pathogens for diagnosis of carbohydrate-based diseases.

1. A fabrication method for carbohydrate chips, which comprises the steps of (i) preparing a solid support derivatized by functional groups to which carbohydrates are attached; (ii) printing unmodified carbohydrate without the linker on the derivatized solid surface; and (iii) reacting said carbohydrates with the solid surface to site-specifically and covalently immobilize the carbohydrates.

2. A fabrication method for carbohydrate chips according to claim 1, wherein the functional group of step (i) is a hydrazide or aminooxy group.

3. A fabrication method for carbohydrate chips according to claim 2, wherein the linkers connected to the hydrazide or aminooxy on the solid surface are selected from the group consisting of hydrocarbon chains inserted by heteroatoms, and of a length exceeding 10 atoms.

4. A fabrication method for carbohydrate chips according to claim 3, wherein the hydrocarbon chains are nonbranched and have from 25 to 55 atoms.

5. A fabrication method for carbohydrate chips according to claim 1, wherein the solid surface is selected from the group consisting of silicon, polymer, mica, plastic, glass, gold, paper, membrane or a combination thereof.

6. A fabrication method for carbohydrate chips according to claim 1, wherein the unmodified carbohydrates are natural or chemically or enzymatically prepared carbohydrates selected from the group consisting of mono-, di-, oligo- and polysaccharides.

7. A fabrication method for carbohydrate chips according to claim 1, wherein printing of said unmodified carbohydrate in step (ii) is carried out by using a micropipette or a microarrayer.

8. A fabrication method for carbohydrate chips according to claim 1, wherein the process for site-specifically and covalently immobilizing said carbohydrate on the solid support in step (iii) is performed by reacting the carbohydrates on the solid surface at a temperature between 40° C. and 60° C. for 8 hours or more.

9. A method for detecting carbohydrates bound to proteins on the carbohydrate chips fabricated according to claim 1, which comprises analyzing with labeled or non-labeled detection systems.

10. A method for detecting carbohydrates according to claim 9, wherein said non-labeled detection system is selected from the group consisting of mass spectrometer and surface plasmon resonance imager.

11. The method for detecting carbohydrates according to claim 9, wherein said labeled detection system are selected from the group consisting of scanning-based instruments and instruments using a CCD camera.

12. The method for detecting carbohydrates according to claim 9, wherein said sample is a human or animal tissue or body fluid, selected from the group consisting of blood, serum, urine, milk, sweat, bone marrow, and lymphatic fluid.

13. The method for detecting carbohydrates according to claim 9, which is employed to detect and diagnose diseases including genetic disorders, cancers, and viral and bacterial infections.

14. A method of diagnosing diseases, which employs the carbohydrate chips fabricated according to claim 1.

15. A method of diagnosing diseases according to claim 14, wherein said disease is selected from the group consisting of genetic disorders, cancer, and viral and bacterial infections.

16. A fabrication method for carbohydrate chips according to claim 2, wherein the solid surface is selected from the group consisting of silicon, polymer, mica, plastic, glass, gold, paper, membrane or a combination thereof.

17. A fabrication method for carbohydrate chips according to claim 3, wherein the solid surface is selected from the group consisting of silicon, polymer, mica, plastic, glass, gold, paper, membrane or a combination thereof.

18. A fabrication method for carbohydrate chips according to claim 4, wherein the solid surface is selected from the group consisting of silicon, polymer, mica, plastic, glass, gold, paper, membrane or a combination thereof.

19. A method of diagnosing diseases, which employs the carbohydrate chips fabricated according to claim 2.

20. A method of diagnosing diseases according to claim 19, wherein said disease is selected from the group consisting of genetic disorders, cancer, and viral and bacterial infections.
